(19) United States
(12) Patent Application Publication
(10) Pub. No.: US 2021/0299010 A1
(43) Pub. Date: Sep. 30, 2021

(54) Skin Protecting Cosmetic Concealer Ointment

(71) Applicant: Derma Jam LLC, Villa Park, CA (US)

(72) Inventor: Kelley Wise, Villa Park, CA (US)

(73) Assignee: Derma Jam LLC, Villa Park, CA (US)

(21) Appl. No.: 17/216,128

(22) Filed: Mar. 29, 2021

Related U.S. Application Data
(60) Provisional application No. 63/148,761, filed on Feb. 12, 2021.
(60) Provisional application No. 63/002,941, filed on Mar. 31, 2020.

(51) Int. Cl. A61K 8/29; A61Q 1/02; A61Q 17/04; A61K 8/27; A61K 8/19; A61K 8/11; A61K 8/02; A61K 8/92

(52) U.S. Cl. CPC A61K 8/29; A61Q 1/02; A61Q 17/04; A61K 8/27; A61K 2800/612; A61K 8/11; A61K 8/0216; A61K 8/922; A61K 2800/412; A61K 8/19

(57) ABSTRACT

A therapeutic ointment is disclosed that is a cosmetic concealer that conceals and protects wounds, burns, rashes, and other skin conditions by incorporating a plurality of anti-microbial metal oxides with iron oxide pigments, petrolatum and tocopherol acetate or a comparable organic carrier emulsion into a skin-tone protective ointment concealer that protects the wound, promotes healing and renders the wound or wounded area less visible or invisible on the skin.

### CROSS-REFERENCE TO PENDING APPLICATIONS

This application claims the benefit of U.S. provisional application 63/148,761 filed Feb. 12, 2021 and entitled “Skin Protecting Cosmetic Concealer Ointment” and U.S. Provisional Application 63/002,941 filed Mar. 31, 2020 and entitled “Skin Protecting Cosmetic Concealer Ointment,” the contents of which are herein all incorporated by reference.

### BACKGROUND OF THE INVENTION

This invention relates to topical ointments, burn creams. In particular, this invention relates to new improved properties to these ointments having advantages over previously known ointments by adding skin tone wound concealing properties to ointments to cover wounds, burns or redness.

It would be advantageous to have an ointment that conceals burns, rashes, or discoloration on skin by incorporating opaque skin-tone pigment into the ointment so as to render a burn or wound on skin less visible by covering the wound with the ointment that contains the opaque skin-tone color pigment that blends with the user's skin to conceal the wound while it is healing.

### SUMMARY OF THE INVENTION

The invention which is an improvement over prior art such as cosmetic concealers such as Dermablend which are not suitable to place on wounded skin, and skin protectants such as Vaseline, Aquaphor, Epicure, and other over the counter ointments and balms used to protect and medicate the skin but do not act as a cosmetic concealer to hide redness, burns, and wounds. This novel invention is an improvement over all other prior art cosmetic concealers, ointments, and skin protectants because it provides cosmetic skin tone concealer properties and also seals, protects and medicates wounded skin as taught in prior art.

There are numerous benefits to this novel invention over prior art. People can experience a wide range of skin conditions and minor wounds that they would prefer not to cover with a bandage and not have the wound or healing treatment area visible to others by only using Aquaphor or other protectant products. Normal cosmetic concealers and makeup products alone are not suitable to put over a wound or healing skin because they do not provide anti-microbial protection. This novel invention combines high levels of anti-microbial titanium dioxide and or zinc oxide with iron oxides and a protective anti-oxidant emulsion matrix to form a unique concealer product that protects wounded skin while it heals.

One of the many benefits can be illustrated by a cancer patient receiving radiation therapy that experiences unsightly burns on their skin from the treatment where ionizing radiation is focused on a targeted area on their body to treat skin cancer and other cancers. These patients apply burn ointment products to the affected area on the skin between radiation therapy treatments. Such products are either clear or white and when applied to the affected area these products make the burned treatment area appear more noticeable and causes the patient to suffer embarrassment and anxiety for having the burned treatment area on their body made more noticeable.

This invention solves this problem by providing a cosmetic that puts a concealing layer of opaque anti-microbial titanium dioxide and or zinc oxide suspended in a skin protecting anti-oxidant emulsion matrix to protect the wounded, healing, or burned area of the skin and hide or conceal the discoloration caused by the wound or burn in one product and with one application. This novel invention is a protective wound healing mineral based concealer that mixes the wound healing properties of titanium dioxide and or zinc oxide with a protective petrolatum emulsion matrix containing a high ratio of anti-oxidant tocopherol acetate oil “Synthetic Vitamin E” to help promote healing and protect the skin against free radicals created by the breakdown of the titanium dioxide and zinc oxide that would be caused by prolonged exposure to sunlight.

An ointment or common wound protectant like Vaseline® petroleum jelly or Aquaphor® healing ointment is recommended by doctors for protecting wounds and soothing burned, damaged skin due to radiation treatments and other burns on the skin. These ointments or protectants serve the purpose of protecting the skin but do not conceal the burn or wounded area. White Petrolatum is commonly used by compounding pharmacists as a base for making skin ointments. Petrolatum is a suitable carrier base for the anti-microbial metal oxides to be suspended onto wounded or burned skin. Petrolatum works as a protective barrier for retaining the skin's natural moisture and to prevent infections to the burn or wound. Prior art wound protection products using Petrolatum such as Aquaphor may contain other components like mineral oil, ceresin, lanolin, panthenol, glycerin, and bisabolol but no opaque metal oxides such as titanium dioxide or zinc oxide and iron oxides that could conceal the wounded or treated area in a matching skin tone.

Petrolatum or a mixture such as Aquaphor® healing ointment containing petrolatum with mineral oil, and glycerin are preferred wound protectants but their gelatinous consistency does not lend itself to acting as the primary component of a wound concealer. An embodiment of the invention in a gelatinous form would not be preferable because it would be more likely to wipe off and make a mess for the user. Therefore the preferred embodiment of the invention is formulated to have the appearance and texture of a concealer.

Cosmetic concealers contain mostly mineral oil, talc, various waxes and only small amounts of titanium dioxide and iron oxides. There is not a high enough concentration of titanium dioxide to provide any therapeutic value or to be efficacious to apply directly to a wound or healing skin. Prior art cosmetic concealers do not protect wounded skin and should not be placed on wounded skin because such products could harbor an unacceptable level of germs being able to grow on the product after use. Our novel invention is an improvement over cosmetic concealers because its high concentration of titanium dioxide and or zinc oxide is anti-microbial, anti-fungal, and anti-viral naturally. Because of the high concentration of titanium dioxide and or zinc oxide in the formula, harmful germs, fungus, and viruses will not grow in the container on the product or on the area of the skin where the product is applied.

In the preferred embodiment of the invention the opaque powder metal oxide components of the formula are greater than the emulsion components and when blended have the physical composition of a solid or semi solid cosmetic concealer type of product. This embodiment of the invention when applied in a small amount to the wounded skin forms a thin protective layer over the wound that attaches to the wounded skin and promotes healing while blending in with the user's natural skin color to cosmetically hide the wound without the product easily wiping off.

The novel concealing formulation of the invention can be additionally applied to other embodiments of the invention including but not limited to using alternative emulsions matrixes to petrolatum and tocopherol acetate in the formula such as organic emulsions, medicated emulsions, liquid emulsions, silicone elastomers, waxes, ointments, salves, balms, or other type of wound care preparations. The metal oxide components in this formula can be blended with an absorption base type of ointment, an Oleaginous Base ointment, hydrophilic base ointment or water soluble ointments.

In the preferred embodiment, of the invention the formula is made by adding colored iron oxides to titanium dioxide and or zinc oxide which is mixed with USP Petrolatum and tocopherol acetate using such methods as a mortar and pestle, or a planetary mixer, or an industrial roller mill to produce a thick semi solid wound concealer product.

The titanium dioxide and zinc oxide are beneficial in many ways to provide antimicrobial properties, promote healing, relieve irritation, sooth inflammation and relieve itchiness on the skin. Because these oxides are opaque white, these ingredients are not acceptable to patients and consumers using existing wound care or skin protecting ointments which contain them. My novel invention contains blends of iron oxide pigments to make the white opaque oxides in the ointment blend into the skin in a protective, anti-oxidant carrier emulsion matrix applied as a cosmetic to the wound.

The preferred embodiment of the invention is highly desirable to patients with chronic visible skin conditions like eczema, and covering minor wounds and scabs, or patients receiving radiation therapy treatments for skin cancer particularly for burn on the face and other noticeable areas on the body that wish to conceal the treatment area. The product also provides skin tone sunburn protection for the wounded area. The invention can be used to cover skin wounds from tattoo removal, laser treatments, and is effective with eczema, dermatitis, scrapes, scabs, and other minor wounds. Anyone skilled in the art would see the benefit of combining an anti-microbial concealing component with any topical wound care products that could serve as the emulsion matrix component of the invention and could incorporate the invention into such products. Depending on packaging and marketing the invention in its described formula herein could be sold to consumers as a cosmetic, or an over-the-counter skin protectant, or a concealing topical ointment, and incorporated into prescription skin products. Besides the objectives and advantages of the preferred embodiment of the invention described above, there are other objectives and advantages also some of which are:
a) To have a convenient cosmetic that is able to safely conceal minor wounds.
b) To use this method to make clear or white ointments or other topical OTC products into skin matching concealers with skin protectant properties.
c) To provide higher self-esteem to cancer and burn patients during treatment and recovery.
d) To speed up recovery and promote fast wound healing.
e) To provide a healthy alternative to putting concealer makeup onto a wound or skin condition.
f) To provide relief from eczema and other chronic skin conditions.
g) To allow the petrolatum based emulsion of organic oil based emulsion to act as a carrier, or matrix, for the titanium dioxide and the zinc oxide to bind to the surface of the skin and provide wound healing and repair, soothe irritation, and relieve itching while providing concealing protective coverage of the wound.
h) To improve the effectiveness of applying titanium dioxide and or zinc oxide when applied to the skin by combining the anti-oxidant properties of tocopherol acetate and vitamin E to the protective carrier emulsion matrix containing the titanium dioxide and or zinc oxide to help prevent skin damage from free radical forming when these oxides are subjected to prolonged sun exposure and promote wound healing.
i) To create healing preparations and medicine to skin wounds with wound concealing properties.
j) To create makeup products designed to treat skin conditions.
k) To treat conditions on the face where an anti-microbial ointments and or treatments is efficacious to relieve infections, styes, acne and other medical conditions which would benefit from treating a wound or condition and covering it in the same product.
l) To treat geriatric skin conditions by protecting and concealing, and helping to heal wounded damaged aging skin.

### DETAILED DESCRIPTION

In an exemplary embodiment of the invention a wound healing skin-tone wound concealer ointment in different skin tones is made using different amounts of colored iron oxides, in the correct ratio and titanium dioxide and or zinc oxide and a protective emulsion matrix made of USP petrolatum and tocopherol acetate which is mixed together using a mortar and pestle, planetary mixer or roller mill into a thoroughly blended flesh tone concealer.

Preferably, the amount of the metal oxide component of the invention is no more than about 60% to 40% ratio added to the carrier emulsion absorption base, or matrix. The formula illustrated herein shows an average of 56% as the preferable amount of metal oxide component to the emulsion matrix carrier component but that could vary according to product requirements and minor differences in the amount of iron oxides needed for the different skin tones of the product.

The oxides and pigments herein are selected from the group consisting of titanium dioxide, zinc oxide, iron oxide, and mixtures thereof, and have an average particle size of from about 100 nm to about 500 nm, preferably from about 200 nm to about 350 nm. The titanium dioxide may be rutile type or anatase type. Rutile Titanium Dioxide is preferred in the preferred embodiment of the Invention because of the preferred lager average particle size. Another embodiment of the invention may use anatase titanium dioxide although rutile and anatase titanium dioxide are both suitable. The ratio of titanium dioxide to zinc oxide may be adjusted to increase the amount of zinc oxide in the mix or one of these may be omitted for a formula with just zinc oxide or just titanium dioxide as the white oxide component in the formula. It is preferable to use both of these oxides in the formula rather than just one or the other, but a cosmetic embodiment of the invention suitable for concealing wounds but sold or marketed as a cosmetic would use only titanium dioxide without zinc oxide. The preferred embodiment of the invention would blend the determined amounts of titanium dioxide and or zinc oxide and iron oxides with a carrier emulsion matrix which is a suitable dermatologically protective liquid such as petrolatum and a viscous vitamin E component such as tocopherol acetate (synthetic vitamin E oil) together with the petrolatum to form a protective concealer suitable for applying to wounded and healing skin. By adding Vitamin E to the carrier emulsion matrix in the form of tocopherol acetate or other organic forms of vitamin E, the skin receives direct anti-oxidant protection from any free radicals created by the degradation of the titanium dioxide or zinc oxide from extended sun exposure. Although the use of non-Nano sized particles of these oxides are considered safe for use in the Sun, the addition of vitamin e oil minimizes the potential skin damage from free radicals.

In a preferred embodiment of the invention described herein, the pigments are iron oxide powders in the colors red, brown and yellow at varying ratios for each skin tone which are blended with titanium dioxide and or zinc oxide with USP petrolatum and tocopherol acetate (synthetic vitamin E oil) in a mortar and pestle, planetary mixer or a roller mill or other industrial mixer for large quantities to create the medicinal concealer compound in flesh tones. Increasing or decreasing the ratios of iron oxide color mixtures and adding other colors like blue or darker brown for example changes the concealer to match different racial skin tones.

The following formulas are examples of the preferred embodiment of the invention using titanium dioxide with petrolatum to achieve four examples of different flesh tone colors made with the following materials.

| Component | Example 1 | Example 2 | Example 3 | Example 4 |
|---|---|---|---|---|
| Titanium Dioxide | 53.57% | 51.785% | 50% | 46.428% |
| Zinc Oxide | 0% | 0% | 0% | 0% |
| Petrolatum | 28.57% | 28.57% | 28.57% | 28.57% |
| Tocopherol Acetate | 14.29% | 14.29% | 14.29% | 14.29% |
| Yellow Iron Oxide | 1.79% | 1.79% | 3.57% | 1.79% |
| Red Iron Oxide | 0.895% | 1.43% | 1.79% | 1.79% |
| Brown Iron Oxide | 0.895% | 1.43% | 0.895% | 7.14% |
| Totals | 100% | 100% | 100% | 100% |

In an exemplary embodiment, the product has a composition of 45-55% Titanium Dioxide, 25-30% Petrolatum, 10-15% Tocopherol Acetate, 1-4% Yellow Iron Oxide, 0.5-2% Red Iron Oxide, and 0.5-10% Brown Iron Oxide.

The following formulas are examples of the preferred embodiment of the invention using titanium dioxide and zinc oxide with organic alternatives to petrolatum to achieve four examples of different flesh tone colors made with the following materials.

| Component | Example 1 | Example 2 | Example 3 | Example 4 |
|---|---|---|---|---|
| Titanium Dioxide | 38.57% | 36.785% | 35% | 31.428% |
| Zinc Oxide | 15% | 15% | 15% | 15% |
| Shea Butter | 28.57% | 28.57% | 28.57% | 28.57% |
| Vitamin E Oil | 14.29% | 14.29% | 14.29% | 14.29% |
| Yellow Iron Oxide | 1.79% | 1.79% | 3.57% | 1.79% |
| Red Iron Oxide | 0.895% | 1.43% | 1.79% | 1.79% |
| Brown Iron Oxide | 0.895% | 1.43% | 0.895% | 7.14% |
| Totals | 100% | 100% | 100% | 100% |

In an exemplary embodiment, the product has a composition of 30-40% Titanium Dioxide, 10-20% Zinc Oxide, 25-30% Shea Butter, 10-20% Vitamin E Oil, 1.5-4% Yellow Iron Oxide, 0.5-2.0% Red Iron Oxide, and 0.5-10% Brown Iron Oxide.

The following formulas are examples of the preferred embodiment of the invention using titanium dioxide without zinc oxide and with examples of organic alternatives to petrolatum to achieve four examples of different flesh tone colors made with the following materials.

| Component | Example 1 | Example 2 | Example 3 | Example 4 |
|---|---|---|---|---|
| Titanium Dioxide | 53.57% | 51.785% | 50% | 46.428% |
| Zinc Oxide | 0% | 0% | 0% | 0% |
| Shea Butter | 28.57% | 28.57% | 28.57% | 28.57% |
| Vitamin E Oil | 14.29% | 14.29% | 14.29% | 14.29% |
| Yellow Iron Oxide | 1.79% | 1.79% | 3.57% | 1.79% |
| Red Iron Oxide | 0.895% | 1.43% | 1.79% | 1.79% |
| Brown Iron Oxide | 0.895% | 1.43% | 0.895% | 7.14% |
| Totals | 100% | 100% | 100% | 100% |

In an exemplary embodiment, the product has a composition of 45-55% Titanium Dioxide, 25-30% Shea Butter, 10-20% Vitamin E Oil, 1.5-4% Yellow Iron Oxide, 0.5-2.0% Red Iron Oxide, and 0.5-10% Brown Iron Oxide.

The following formulas are examples of the preferred embodiment of the invention using titanium dioxide and zinc oxide with organic alternatives to petrolatum to achieve four examples of different flesh tone colors made with the following materials.

| Component | Example 1 | Example 2 | Example 3 | Example 4 |
|---|---|---|---|---|
| Titanium Dioxide | 38.57% | 36.785% | 35% | 31.428% |
| Zinc Oxide | 15% | 15% | 15% | 15% |
| Shea Butter | 28.57% | 28.57% | 28.57% | 28.57% |
| Vitamin E Oil | 14.29% | 14.29% | 14.29% | 14.29% |
| Yellow Iron Oxide | 1.79% | 1.79% | 3.57% | 1.79% |
| Red Iron Oxide | 0.895% | 1.43% | 1.79% | 1.79% |
| Brown Iron Oxide | 0.895% | 1.43% | 0.895% | 7.14% |
| Total | 100% | 100% | 100% | 100% |

In an exemplary embodiment, the product has a composition of 30-40% Titanium Dioxide, 10-20% Zinc Oxide, 25-30% Shea Butter, 10-20% Vitamin E Oil, 1.5-4% Yellow Iron Oxide, 0.5-2.0% Red Iron Oxide, and 0.5-10% Brown Iron Oxide.

The following formulas are examples of the preferred embodiment of the invention using titanium dioxide without zinc oxide and with examples of organic alternatives to petrolatum to achieve four examples of different flesh tone colors made with the following materials.

| Component | Example 1 | Example 2 | Example 3 | Example 4 |
|---|---|---|---|---|
| Titanium Dioxide | 53.57% | 51.785% | 50% | 46.428% |
| Zinc Oxide | 0% | 0% | 0% | 0% |
| Shea Butter | 28.57% | 28.57% | 28.57% | 28.57% |
| Vitamin E Oil | 14.29% | 14.29% | 14.29% | 14.29% |
| Yellow Iron Oxide | 1.79% | 1.79% | 3.57% | 1.79% |
| Red Iron Oxide | 0.895% | 1.43% | 1.79% | 1.79% |
| Brown Iron Oxide | 0.895% | 1.43% | 0.895% | 7.14% |
| Totals | 100% | 100% | 100% | 100% |

In an exemplary embodiment, the product has a composition of 45-55% Titanium Dioxide, 25-30% Shea Butter, 10-20% Vitamin E Oil, 1.5-4% Yellow Iron Oxide, 0.5-2.0% Red Iron Oxide, and 0.5-10% Brown Iron Oxide.

The above formulations may be prepared as follows: The wound concealer is mixed and thoroughly blended in a mortar and pestle, planetary mixer, or roller mill or other system normally used to mix a semi solid cosmetic product and then injected or pressed into to container. Test samples were prepared with a planetary mixer with a stainless steel bowl with blade mixer. The mixture was then transferred to an extrusion filling system similar to a cookie press that contained the mixture in a metal tube and a piston plunger pressed a measured amount of the mixture through a ½″ diameter hole into each one-ounce round metal tin container and sealed with a screw top lid.

Cleaning, sanitizing and drying was done on the stainless mixing bowl and mixing blade as well as other stainless-steel bowls, measuring cup, stainless steel spoons, long flat spatulas and the extrusion filling system. The preparation area and preparation table were cleaned and sanitized. A heavy-duty planetary mixer was placed on the table with the scale, the mixing bowls. A precision digital scale was used to measure out the ingredients by weight and place the measured ingredients into stainless steel bowls. Additional steps were also take to ensure the product was uncontaminated such as wearing N95 mask, face shield, and rubber gloves when measuring the ingredients, mixing the ingredients, and packing the competed mixture into the containers.

To make fifty ounces of the formula as one example in the color Bisque in the cosmetic form of the invention without the zinc oxide included, 725 grams of purified titanium dioxide was measured out, but if zinc oxide were to be used it would also be a ratio of 60% titanium dioxide and 40% zinc oxide or a variation thereof equaling 725 grams rather than 725 grams of just titanium dioxide or just zinc oxide.

25 grams of cosmetics grade yellow iron oxide was measured, 40 grams of brown iron oxide, and 40 grams of red iron oxide. 400 grams of medical grade white petrolatum USP was measured out onto a flat tray, and 200 grams of tocopherol acetate oil in the measuring cup. Tocopherol acetate was poured into the mixing bowl and then the measured iron oxides and titanium oxide was poured into the 6 quart mixing bowl. The petrolatum was then placed in the bowl on top of the powder and the mixer was started. The bowl and blender blade was scraped during the process with the long stainless spatulas and the mixture was blended completely for thirty minutes at room temperature (20 to 25 Celsius). The mixed formula was then transferred from the mixing bowl to a covered sanitized stainless steel bowl or container to wait for the packaging or filling process. The mixture was placed into the extrusion filling device tube and a piston plunger in the device pushed the mixture out the hole into a round tin that was below and in close proximity to the extruder device orifice in measured amounts into the container which was then sealed and labeled.

The same mixing process and the filling process described herein would be also used to make any other skin tone color variations by using different ratios of the iron oxides. The formula examples shown herein only produce four colors but other variations in the ratio of the pigments would create a much wider variety of colors which is anticipated and intended in the invention described. Based on the disclosure herein, anyone skilled in the art could create a wide variety of skin tones beyond the examples contained herein. The organic version of the invention can also be made using this method by substituting the petrolatum and tocopherol acetate in the formula with organic ingredients like Shea butter and vitamin e oil, or similar ingredients like jojoba oil and Aloe Vera. The formula can be packaged into squeeze tubes or other containers by minimally heating the blended formula (40 to 45 Celsius) to allow for mechanically rapid filling open ended squeeze tubes using a filling system other than an extrusion type of filling system.

Additional formulations may be made in accordance with the process set forth in these Examples in order to create compositions according to this invention which may contain one or more active ingredients in addition. The ingredients of the example formulation could be substituted with a different carrier other than white petrolatum or any of the carriers contemplated in the forgoing examples. Another embodiment of the invention as an emulsion could be achieved with water-based extracts and organic oils such as Shea butter, Cannabis Oils, or synthetic oils not based on petrolatum for an ointment base. Another embodiment of the invention could be achieved with a wax-based balm as well by adding the concealing component described in the invention.

The carrier ointment base combined with the concealer component of this invention shown in the examples provides a cosmetically concealing composition of skin protection ointment and can be formulated with additional topical active materials, as set forth to achieve desired therapeutic results for the skin with the concealer properties of a cosmetic concealer. The following formulations may be produced according to the procedure set forth above in the example formulation with the understanding that the process may be adjusted to suit the specific materials. Adjustments may include, but are not limited to, pH of the final product, order of addition of raw materials and/or processing temperatures as necessary. The stability of this ointment was shown in tests of one month at room temperature, 37° C. and 45° C. with no loss of homogeneity was observed.

The foregoing example of the invention includes the addition of zinc oxide, titanium dioxide and iron oxides to create a skin tone opaque appearance on the skin when mixed in an emulsion matrix with a petrolatum or organic oil based medicated ointment formulation and it will be obvious to those skilled in the art that other medicated ointments and protectant balms and creams such as Aquaphor, Epicure, Vaseline, and other skin balms and ointments could be used as the emulsion matrix component of the invention in order to add the concealing properties of the invention to other applications or products. In addition other suitable medicaments could be used in the formula to add healing properties within FDA guidelines such as, hydrocortisone, iodochlorhydroxypryin, lidocaine, pyrilamine maleate salicylic acid, allantoin, resorcinol, chlorhydroxyquinoline, bufexamic acid might be added.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than the foregoing description, and all changes which come within the meaning and range of equivalency of the claims are therefore intended to be embraced therein.

I claim:

1. A component of an ointment formula, comprising
a plurality of particles having diameters in the size range of about 100 nm to 500 nm;
a matrix in combination with a skin matching color;
a UV-attenuating, cosmetically concealing compound; and
a plurality of metal oxide pigment particles being and dispersible within a dermatologically acceptable carrier.

2. The component of claim 1, wherein the plurality of metal oxide pigment particles comprises a coloring agent.

3. The component of claim 1, wherein the plurality of metal oxide pigment particles comprises a plurality of titanium dioxide particles.

4. The component of claim 1, wherein the plurality of metal oxide pigment particles comprises a plurality of zinc oxide particles.

5. The component of claim 1, wherein the plurality of metal oxide pigment particles comprises a plurality of iron oxide particles.

6. The component of claim 1 wherein the plurality of metal oxide pigment particles further comprises a second UV-attenuating compound that is incorporated into said matrix.

7. The component of claim 1 wherein the plurality of metal oxide pigment particles comprises a first UV-attenuating compound and a second UV-attenuating compound that are individually incorporated into said matrix to form separately encapsulated first and second UV-attenuating compounds.

8. The component of claim 1 wherein the plurality of metal oxide pigment particles comprises a cosmetically concealing compound that are incorporated into said skin protective ointment compounds.

9. The component of claim 1, said dermatological carrier is a medicated ointment containing one or more medicated ingredients.

10. The component of claim 1, wherein the plurality of metal oxide pigment particles comprises a plurality of titanium dioxide, zinc oxide, yellow iron oxide, red iron oxide and brown iron oxide particles, wherein the component has a composition of 30-40% titanium dioxide, 10-20% zinc oxide, 0.5-4% yellow iron oxide, 0.5-2.0% red iron oxide, and 0.5-10% brown iron oxide.

11. A component of an ointment formula, comprising
a plurality of particles having diameters in the size range of about 100 nm to 500 nm formed in a matrix comprising petrolatum-based emulsion or organic oil-based emulsion; and
a cosmetically concealing compound incorporated into said petrolatum or suitable carrier, said particles being dispersible within a dermatologically acceptable liquid carrier.

12. The component of claim 11, wherein the plurality of metal oxide pigment particles comprises a coloring agent.

13. The component of claim 11, wherein the plurality of metal oxide pigment particles comprises a plurality of titanium dioxide particles.

14. The component of claim 11, wherein the plurality of metal oxide pigment particles comprises a plurality of zinc oxide particles.

15. The component of claim 11, wherein the plurality of metal oxide pigment particles comprises a plurality of iron oxide particles.

16. The component of claim 11 wherein the plurality of metal oxide pigment particles further comprises a second UV-attenuating compound that is incorporated into said matrix.

17. The component of claim 11 wherein the plurality of metal oxide pigment particles comprises a first UV-attenuating compound and a second UV-attenuating compound that are individually incorporated into said matrix to form separately encapsulated first and second UV-attenuating compounds.

18. The component of claim 11 wherein the plurality of metal oxide pigment particles comprises a cosmetically concealing compound that are incorporated into said skin protective ointment compounds.

19. The component of claim 11, said dermatological carrier is a medicated ointment containing one or more medicated ingredients.
